(19) United States
(12) Patent Application Publication
(10) Pub. No.: US 2022/0251091 A1
(43) Pub. Date: Aug. 11, 2022

(54) AMORPHOUS UMBRALISIB MONOTOSYLATE

(71) Applicant: Johnson Matthey Public Limited Company, London (GB)

(72) Inventor: Na YAO, Devens, MA (US)

(21) Appl. No.: 17/597,634

(22) PCT Filed: Jul. 15, 2020
(86) PCT No.: PCT/GB2020/051701

§ 371 Date: Jan. 14, 2022

Related U.S. Application Data
(60) Provisional application No. 62/874,207, filed on Jul. 15, 2019.

(51) Int. Cl. C07D 487/04; A61P 35/00

(52) U.S. Cl. CPC C07D 487/04; A61P 35/00

(57) ABSTRACT

The present disclosure is directed to amorphous umbralisib monotosylate, and to processes for its preparation; pharmaceutical compositions comprising amorphous umbralisib monotosylate; and to a method for treating a patient using amorphous umbralisib monotosylate.

### FIELD OF THE DISCLOSURE

The present disclosure relates to an amorphous form of umbralisib monotosylate and to methods of preparing the same. The present disclosure also relates to pharmaceutical compositions comprising the form and methods for treating disease using the form.

### BACKGROUND OF THE DISCLOSURE

Umbralisib, having the chemical designation (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one, is an orally available PI3K delta inhibitor. Umbralisib has the following structure:

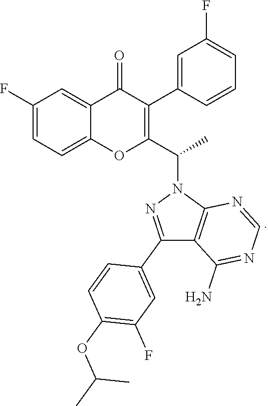 [SMILES: CC(C)Oc1ccc(-c2nn([C@@H](C)c3oc4ccc(F)cc4c(=O)c3-c3cccc(F)c3)c3ncnc(N)c23)cc1F]

Inhibition of PI3K delta signaling with umbralisib has demonstrated activity in several pre-clinical models and primary cells from patients with hematologic malignancies. In a Phase 2 trial, umbralisib provided effective PI3K-delta inhibition and appeared well-tolerated among patients with relapsed/refractory marginal zone lymphoma. Umbralisib is currently in Phase 3 clinical development in combination with ublituximab for patients with hematologic malignancies. Hematologic malignancies are forms of cancer that begin in the cells of blood-forming tissue, such as the bone marrow, or in the cells of the immune system. Examples of hematologic cancer are acute and chronic leukemias, lymphomas, multiple myeloma and myelodysplastic syndromes. Lymphomas can include follicular lymphoma (FL), small lymphocytic lymphoma (SLL), non-Hodgkin lymphoma (NHL), and diffuse large B-cell lymphoma (DLBCL), among others. Leukemia can include chronic lymphocytic leukemia (CLL), among others. The U.S. Food and Drug Administration (FDA) has granted orphan drug designation to umbralisib for the treatment of patients with follicular lymphoma and for the treatment of patients with nodal, extranodal, and splenic marginal zone lymphoma.

U.S. Pat. No. 9,150,579 discloses umbralisib and pharmaceutically acceptable salts thereof, such as 4-methylbenzenesulfonate (also known as tosylate), sulphate, hydrochloride, benzenesulfonate, maleate, and camphor sulfonate salts. U.S. Pat. Nos. 9,969,740 and 10,414,773 and U.S. Patent Application Publication No. 2019/0382411 disclose solid state forms of a p-toluenesulfonic acid salt (PTSA) of umbralisib. None of these references disclose an amorphous form of umbralisib monotosylate.

An amorphous form of a compound is considered to be a solid state form that lacks long-range order relative to crystalline solid state forms of the compound. The amorphous form is chemically identical to other crystalline solid state forms but can exhibit different physical properties such as intrinsic solubility, rate of dissolution, density, mechanical property, chemical and physical stability, hygroscopicity, and morphology. The differences in intrinsic solubility also may lead to a difference in the rate of absorption, thus impacting bioavailability. Generally, amorphous compounds have a higher solubility than crystalline compounds.

### SUMMARY OF THE DISCLOSURE

The present invention is directed to an amorphous form of umbralisib monotosylate. The present invention is further directed to processes for the preparation of an amorphous form of umbralisib monotosylate. The present invention also is directed to pharmaceutical compositions comprising amorphous umbralisib monotosylate, and a method for treating disease using amorphous umbralisib monotosylate.

### BRIEF DESCRIPTION OF THE DRAWINGS

FIG. 1 provides a representative XRPD pattern of amorphous umbralisib monotosylate prepared by dry grinding, expressed in terms of ° 20.

FIG. 2 provides a representative mDSC plot of amorphous umbralisib monotosylate prepared by dry grinding.

FIG. 3 provides a representative DVS plot of amorphous umbralisib monotosylate prepared by dry grinding.

FIG. 4 provides representative sorption isotherm results of amorphous umbralisib monotosylate prepared by dry grinding.

FIG. 5 provides a representative XRPD pattern of amorphous umbralisib monotosylate prepared by dry grinding, after DVS, expressed in terms of ° 20.

FIG. 6 provides a representative XRPD pattern of amorphous umbralisib monotosylate prepared by evaporation in methanol, expressed in terms of ° 20.

FIG. 7 provides a representative mDSC plot of amorphous umbralisib monotosylate prepared by evaporation in methanol.

FIG. 8 provides a representative TGA plot of amorphous umbralisib monotosylate prepared by evaporation in methanol.

FIG. 9 provides a representative DVS plot of amorphous umbralisib monotosylate prepared by evaporation in methanol.

FIG. 10 provides representative sorption isotherm results of amorphous umbralisib monotosylate prepared by evaporation in methanol.

FIG. 11 provides a representative XRPD pattern of amorphous umbralisib monotosylate prepared by evaporation in methanol, after DVS, expressed in terms of ° 20.

FIG. 12 provides a representative ¹H-NMR plot of amorphous umbralisib monotosylate prepared by evaporation in methanol.

FIG. 13(a) provides a representative FTIR spectra for amorphous umbralisib monotosylate prepared by evaporation in methanol and FIG. 13(b) provides a representative FTIR spectra for a crystalline form of umbralisib monotosylate.

FIG. 14 provides a representative XRPD pattern of amorphous umbralisib monotosylate prepared by evaporation in methanol, after storage at 40° C. under vacuum conditions for 2 weeks.

FIG. 15 provides a representative mDSC of amorphous umbralisib monotosylate prepared by evaporation in methanol, after storage at 40° C. under vacuum conditions for 2 weeks.

FIG. 16 provides a representative XRPD pattern of amorphous umbralisib monotosylate prepared according to Example 3, expressed in terms of ° 20.

### DETAILED DESCRIPTION OF THE DISCLOSURE

The following description is presented to enable a person of ordinary skill in the art to make and use the various embodiments. Descriptions of specific devices, techniques, and applications are provided only as examples. Various modifications to the examples described herein will be apparent to those of ordinary skill in the art, and the general principles described herein may be applied to other examples and applications without departing from the spirit and scope of the various embodiments. Therefore, the various embodiments are not intended to be limited to the examples described herein and shown but are to be accorded the scope consistent with the claims.

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “pharmaceutical composition” is intended to encompass a pharmaceutically effective amount of the amorphous umbralisib monotosylate and at least one pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, the term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

As used herein and unless otherwise specified, the term “patient” refers to an animal, preferably a mammal, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/or prevented, but has been deemed by a physician, clinician or other medical professional to be at risk for developing said disorder, disease or condition.

As used herein and unless otherwise specified, the terms “polymorph,” “polymorphic form” or related term herein, refer to a crystal form of a molecule, or salt thereof that can exist in two or more forms, as a result of different arrangements or conformations of the molecule or salt thereof of ions in the crystal lattice of the polymorph.

As used herein and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a compound provided herein, with or without other additional active agents, after the onset of symptoms of the particular disease.

Techniques for characterizing crystal and amorphous forms include but are not limited to differential scanning calorimetry (DSC), thermal gravimetric analysis (TGA), dynamic vapor sorption (DVS), X-ray powder diffractometry (XRPD), proton nuclear magnetic resonance (¹H-NMR), Fourier transform infrared spectroscopy (FTIR Spectroscopy), and Optical Microscopy.

Modulated DSC (mDSC) data are collected using a TA Instruments Q2000 DSC. Approximately, samples (2-5 mg) are placed in sealed hermetic aluminum sample pans and mDSC is carried out with modulation ±0.5° C. every 60 s and measured from about 5° C. to about 300° C. at the heating rate of about 1.5° C./min under a nitrogen purge of about 50 mL/min.

TGA data are collected using a TA Instruments TGA Q500. Approximately, samples (2-5 mg) are placed in an open, pre-tared aluminum sample pan and scanned from about 25 to about 350° C. at a rate of about 10° C./min using a nitrogen purge at about 60 mL/min.

XRPD patterns are obtained using a Bruker D8 Advance equipped with a Cu Kα radiation source (λ=1.54 Å), a 9-position sample holder and a LYNXEYE super speed detector. Samples are placed on zero-background, silicon plate holders for analysis. One skilled in the art would recognize that the ° 20 values and the relative intensity values are generated by performing a peak search on the measured data and the d-spacing values are calculated by the instrument from the ° 20 values using Bragg's equation. One skilled in the art would further recognize that the relative intensity for the measured peaks may vary as a result of sample preparation, orientation and instrument used, for example.

¹H-NMR data are collected using a Bruker Ascend 600 MHz NMR equipped with TopSpin software. Samples are prepared by dissolving the compound in deuterated dimethylsulfoxide with 0.05% (v/v) tetramethylsilane (TMS). Spectra are collected at 298 K. The number of scans is 16 for ¹H-NMR.

FTIR spectra are collected using a Thermo Scientific Nicolet iS5. Samples are ground with a mortar and pestle into a mixture of 95% KBr, then pressed by hand into pellets using a Little-Press KBr Pellet Die Kit. The number of scans is 16 for each measurement.

DVS samples (5-10 mg) are analyzed using a TA Instruments Q5000 SA powerful dynamic vapor sorption analyzer. The relative humidity is started at about 50% and adjusted to between about 0-90% humidity with about a 10% increase or decrease in humidity for each step for 3 cycles. The weight of the sample is continuously monitored and recorded.

The present disclosure is directed to amorphous umbralisib monotosylate, processes for the preparation of amorphous umbralisib monotosylate, pharmaceutical compositions comprising amorphous umbralisib monotosylate, and its use for treating a patient with a physiological condition in need of treatment, as herein described in detail.

In one embodiment, the process for preparing amorphous umbralisib monotosylate first comprises the preparation of umbralisib tosylate salt by reactive crystallization of umbralisib free base with p-toluenesulfonic acid (PTSA) in ethyl acetate. In an embodiment, the umbralisib free base and p-toluenesulfonic acid are present in about a 1:1 ratio. In one embodiment, umbralisib free base and p-toluenesulfonic acid are each dissolved separately in ethyl acetate. The two solutions are then mixed together and stirred at room temperature for a period of time. For example, the mixed solution is stirred overnight (about 8-12 h) to effect precipitation of crystalline umbralisib tosylate salt. If precipitation does not occur, the mixed solution may be transferred to a lower temperature, for example to about 5° C., to assist in the formation of a precipitate. For a larger scale, for example, greater than about 800 mg, seeds can be added to the mixed solution to induce the reaction. A solid crystalline umbralisib tosylate salt is obtained by filtration. In another embodiment, the crystalline umbralisib tosylate salt obtained according to the above procedure (Form I) is dried under vacuum at about 40° C. in an oven for a period of time, for example, for about 3 days, to remove any residual ethyl acetate.

In one embodiment of the invention, amorphous umbralisib monotosylate can be prepared from crystalline umbralisib tosylate salt, for example using the crystalline salt prepared by above process, or alternatively from any crystalline umbralisib tosylate salt known in the art, for example, Form A or Form B disclosed in U.S. Pat. No. 10,414,773. In a particular embodiment, the process of the invention comprises
a) grinding crystalline umbralisib tosylate salt, preferably dried, using a mortar and pestle to yield amorphous umbralisib monotosylate.
   In a further embodiment, the grinding is conducted for about 3 minutes.

In yet another embodiment, the process of the invention comprises
a) dissolving crystalline umbralisib tosylate salt in a solvent, and
b) evaporating the solvent to yield amorphous umbralisib monotosylate.
   In a further embodiment of the invention, the dissolving is carried out at an elevated temperature, for example, about 50° C. In an embodiment of the invention, the solvent is an alcoholic solvent; more particularly, the solvent is methanol. In another embodiment, the solvent is evaporated at an elevated temperature under vacuum; more particularly, the solvent is evaporated under vacuum at about 40° C. in an oven overnight (about 8-12 h).

In another embodiment, the process of the invention comprises
a) forming umbralisib monotosylate in solution (without isolation of crystalline umbralisib tosylate salt); and
b) evaporating the solution to yield amorphous umbralisib monotosylate.
   In a further embodiment, umbralisib free base and p-toluenesulfonic acid (PTSA) are dissolved in solution. In a particular embodiment, the solution is an C₁₋₃ alcohol; more particularly, methanol. In one embodiment, the umbralisib free base and PTSA are present in a 1:1 ratio. In a particular embodiment, the umbralisib free base and p-toluenesulfonic acid are each separately dissolved in methanol and then the two methanolic solutions are mixed together. In yet another embodiment, the umbralisib free base and p-toluenesulfonic acid are dissolved in methanol at an elevated temperature, for example, at about 50° C. In a further embodiment, the mixed solution is stirred for about 1-3 h. In one embodiment, the mixed solution is first stirred for about 1-3 h at room temperature and then stirred at about 4° C. overnight for about 8-12 h. In a further embodiment, the evaporating is carried out at about 40° C. in a vacuum oven overnight for about 8-12 h.

An embodiment of the invention is directed to amorphous umbralisib monotosylate. A further embodiment of the invention is amorphous umbralisib monotosylate prepared by a process embodiment as described herein.

Amorphous umbralisib monotosylate is a white solid identified as amorphous by XRPD. The amorphous umbralisib monotosylate obtained by dry grinding has a glass transition temperature (T_{g}) of about 51° C. Amorphous umbralisib monotosylate obtained using crystalline umbralisib tosylate salt as the starting material and evaporating from methanol has a T_{g} of about 75° C. Thus, the T_{g} of amorphous umbralisib monotosylate can vary depending upon its preparation method.

In an embodiment of the invention, amorphous umbralisib monotosylate is stored under dry conditions, for example, under vacuum, in the presence of a desiccant, or with low levels of humidity, for example, about 15% or less.

The present disclosure also encompasses pharmaceutical compositions comprising amorphous umbralisib monotosylate and a pharmaceutically acceptable excipient. Pharmaceutical compositions containing amorphous umbralisib monotosylate may be prepared according to any method known in the art.

The present disclosure also provides for a method of treating disease by administering to a patient, in need thereof, pharmaceutical compositions comprising amorphous umbralisib monotosylate. Diseases intended to be treated include hematological malignancies including, but not limited to, chronic lymphocytic leukemia (CLL), diffuse large B-cell lymphoma (DLBCL), non-Hodgkin lymphoma (NHL), lymphoid lineage, B-cell lymphoma, T-cell lymphoma, Hodgkin's lymphoma, hairy cell lymphoma, Burkett's lymphoma, hematopoietic tumors of myeloid lineage, multiple myelomas, smoldering multiple myeloma, nonsecretory myeloma, osteosclerotic myeloma, plasma cell leukemia, solitary plasmacytoma, extramedullary plasmacytoma, Multiple Myeloma (MM), Small Lymphocytic Lymphoma (SLL), Indolent Non-Hodgkin's Lymphoma (I-NHL), mantle cell lymphoma (MCL), follicular lymphoma, and Waldestrom's macroglobulinemia (WM). Umbralisib may be administered, simultaneously or sequentially, with another anti-cancer agent.

The dosage of the pharmaceutical compositions may be varied over a wide range. Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

### EXAMPLES

Examples 1-3, which follow herein, provide embodiments of the preparation of amorphous umbralisib monotosylate.

### Example 1

### Preparation of Amorphous Umbralisib Monotosylate by Dry Grinding of Crystalline Umbralisib Tosylate Salt

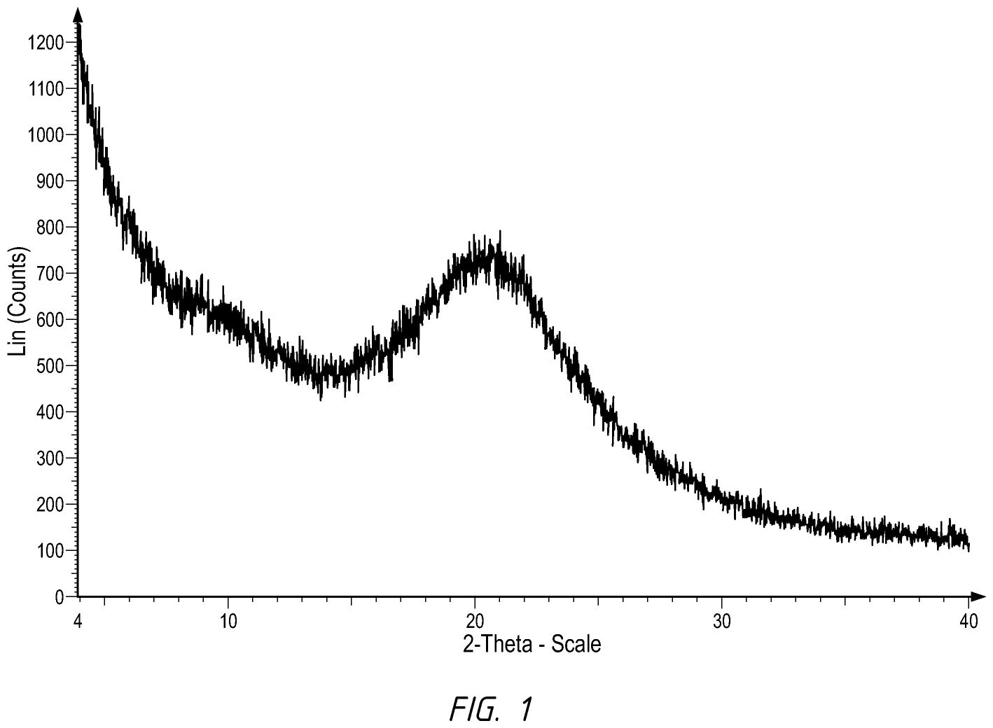

Form I of umbralisib tosylate salt is dried under vacuum at about 40° C. in an oven for at least about 3 days to remove any residual ethyl acetate. About 30 mg of the dried umbralisib tosylate salt is ground manually using a mortar (about 6 cm in diameter) and pestle for about 3 minutes. The ground umbralisib tosylate salt is identified as being amorphous by XRPD. FIG. 1 is a representative XPRD pattern for amorphous umbralisib monotosylate prepared according to Example 1.

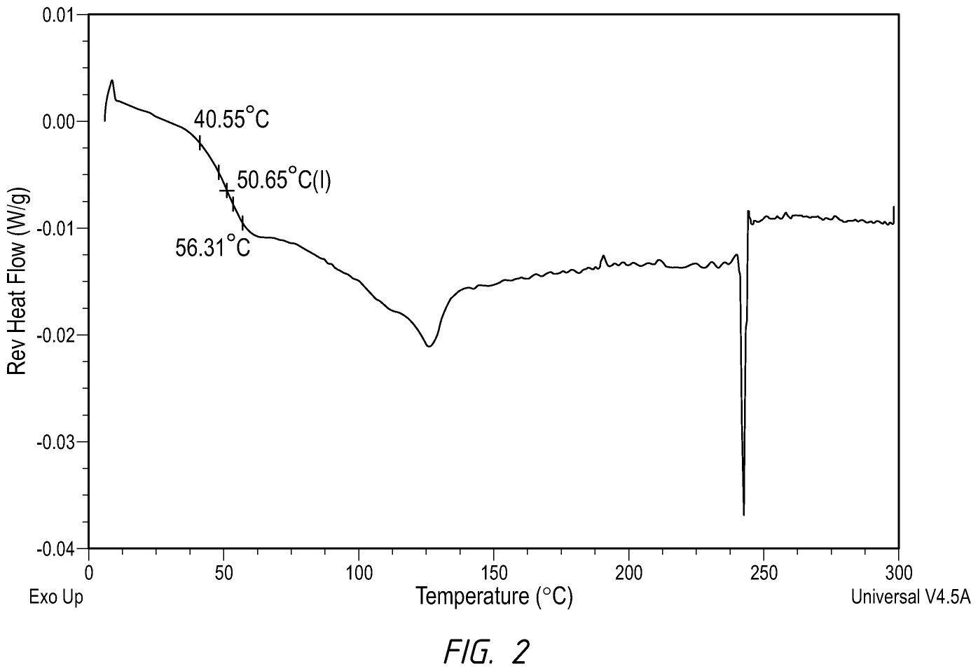

The amorphous umbralisib monotosylate prepared according to Example 1 is characterized by a T_{g} of about 51° C., as depicted in the mDSC thermogram contained in FIG. 2.

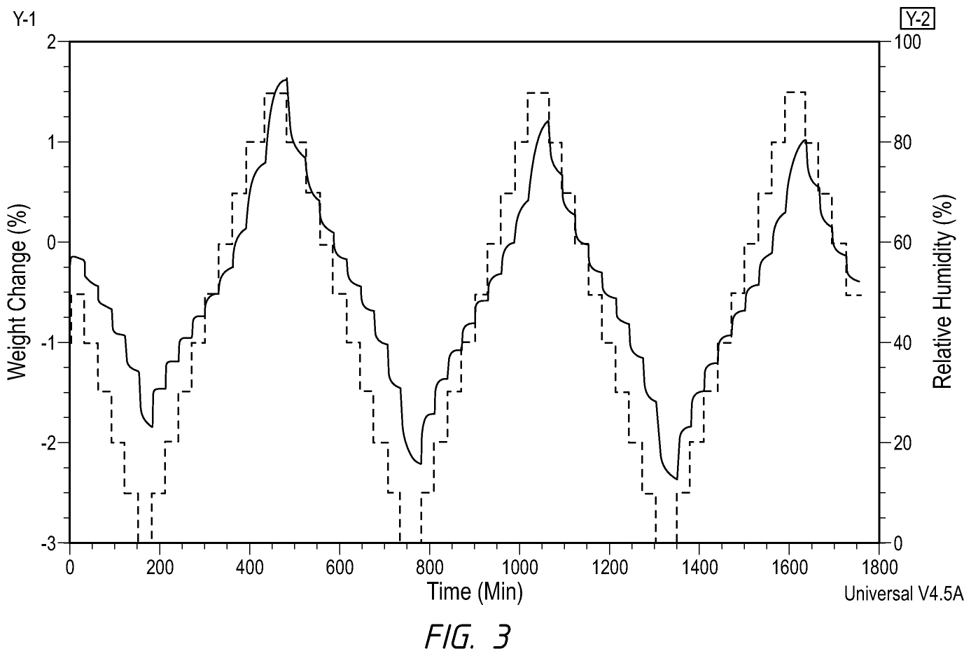

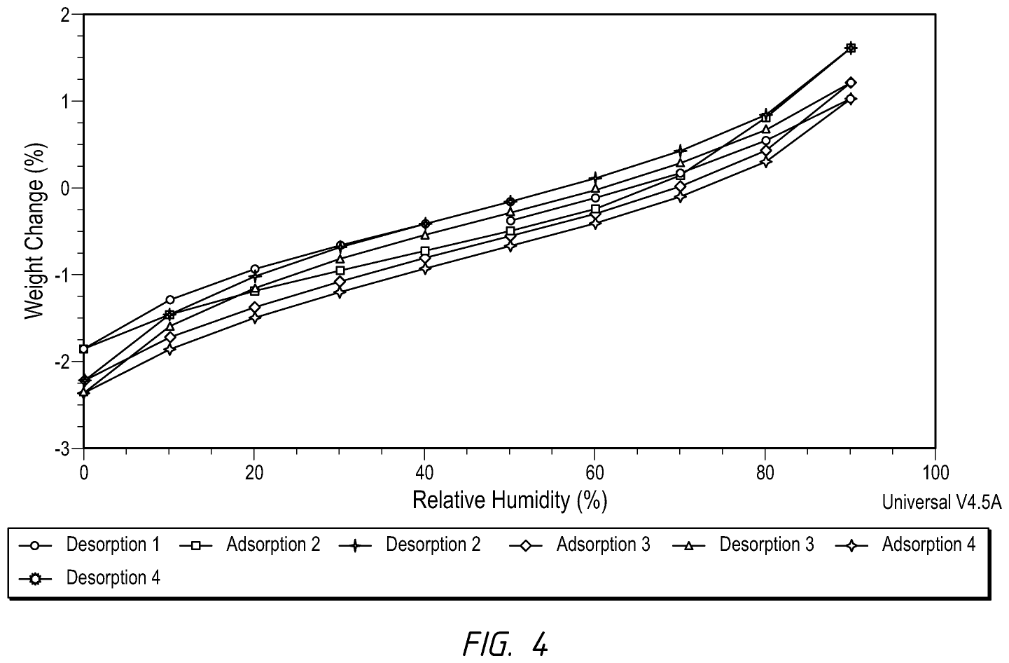

A DVS of amorphous umbralisib monotosylate prepared according to Example 1 indicates the sample is hygroscopic, with about a 4% weight change between about 0-90% relative humidity, as depicted in FIG. 3, and less than about a 1% weight change in the sample over three cycles, as depicted in FIG. 4.

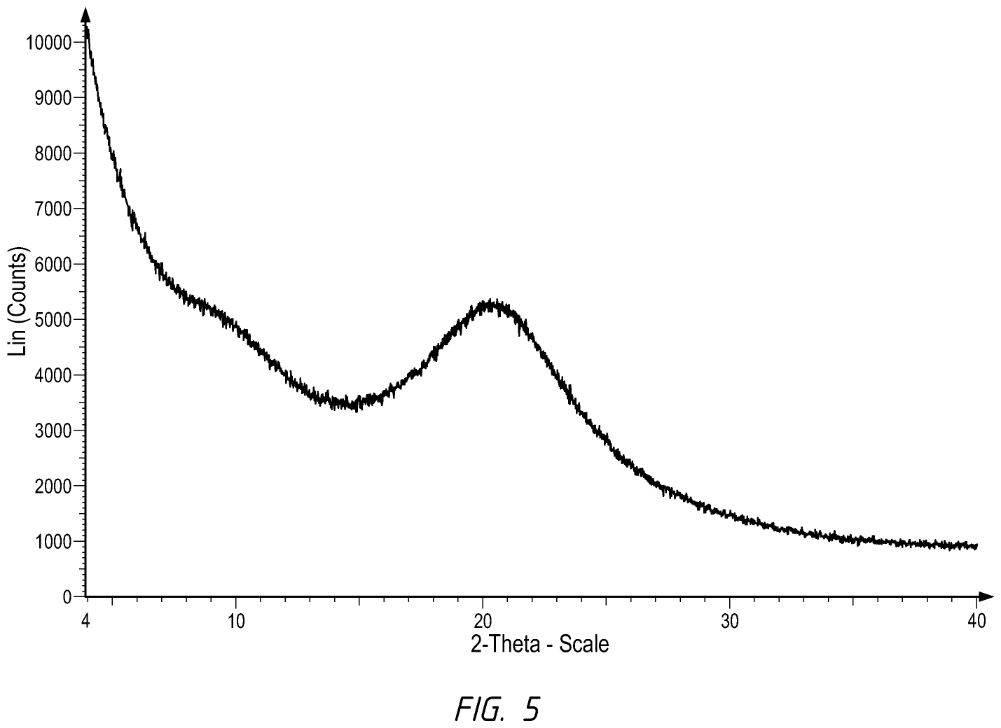

An XRPD pattern of the sample after DVS indicates that the sample is still amorphous, as depicted in FIG. 5.

### Example 2

### Preparation of Amorphous Umbralisib Monotosylate by Dissolution of Crystalline Umbralisib Tosylate Salt in Methanol and its Evaporation Therefrom

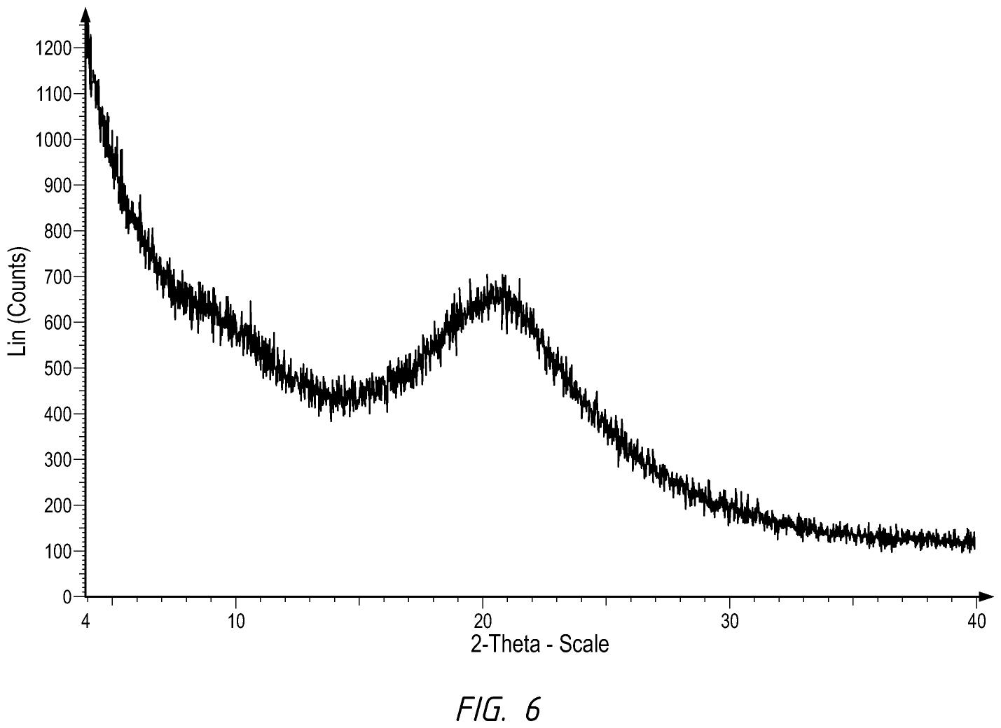

About 470 mg of Form I of umbralisib tosylate salt is dissolved in about 20 mL of methanol at about 50° C. A solid umbralisib tosylate salt is obtained by evaporation of the solution under vacuum at about 40° C. in an oven overnight. The isolated product is identified as being amorphous umbralisib monotosylate by XRPD. FIG. 6 is a representative XPRD pattern for amorphous umbralisib monotosylate prepared according to Example 2.

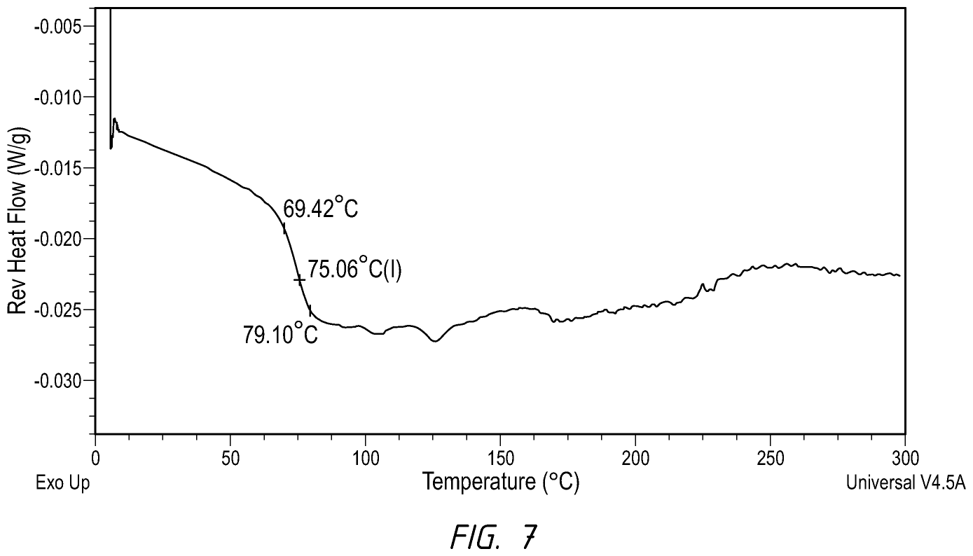

The amorphous umbralisib monotosylate prepared according to Example 2 is characterized by a T_{g} of about 75° C., as depicted in the mDSC thermogram contained in FIG. 7.

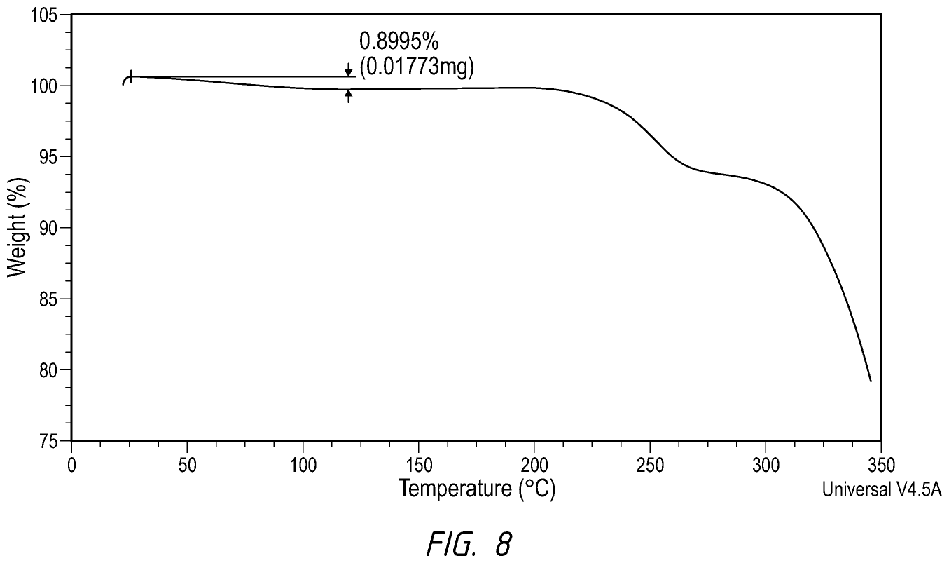

A TGA of amorphous umbralisib monotosylate prepared according to Example 2 shows about a 0.9% weight loss up to about 120° C., as depicted in FIG. 8.

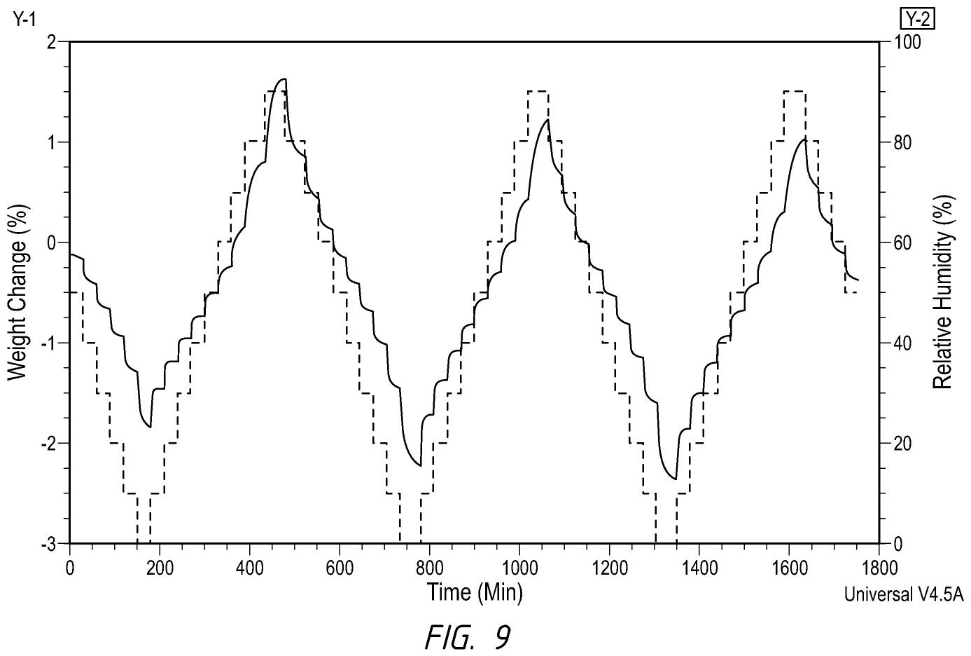

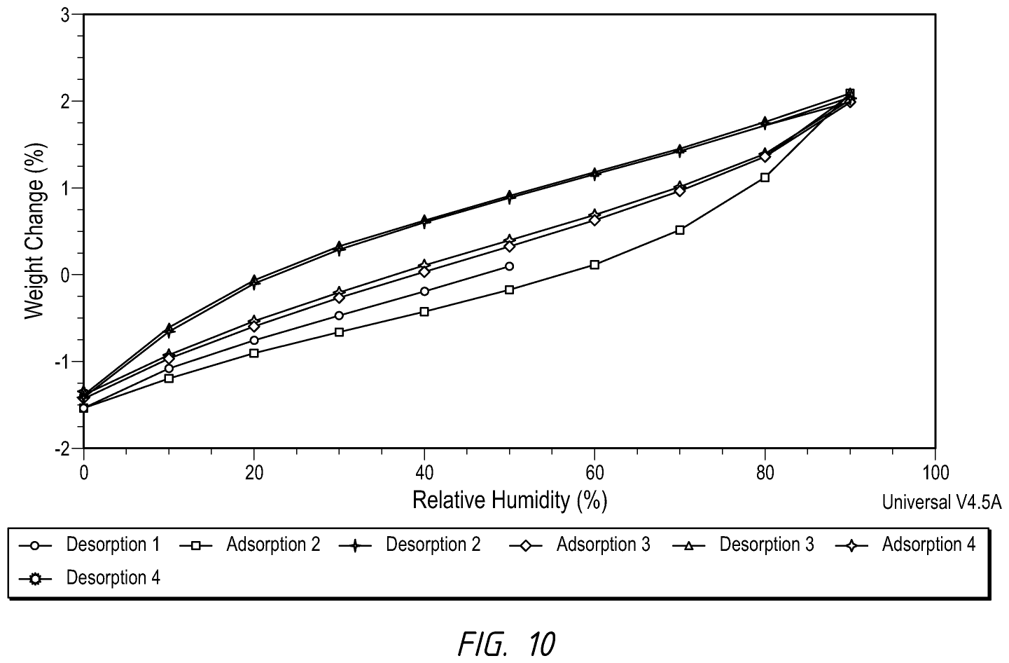

A DVS of amorphous umbralisib monotosylate prepared according to Example 2 indicates that the sample is hygroscopic, with about a 4% weight change between about 0-90% relative humidity, as depicted in FIG. 9, with about a 0.5% weight change in the sample over three cycles, as depicted in FIG. 10.

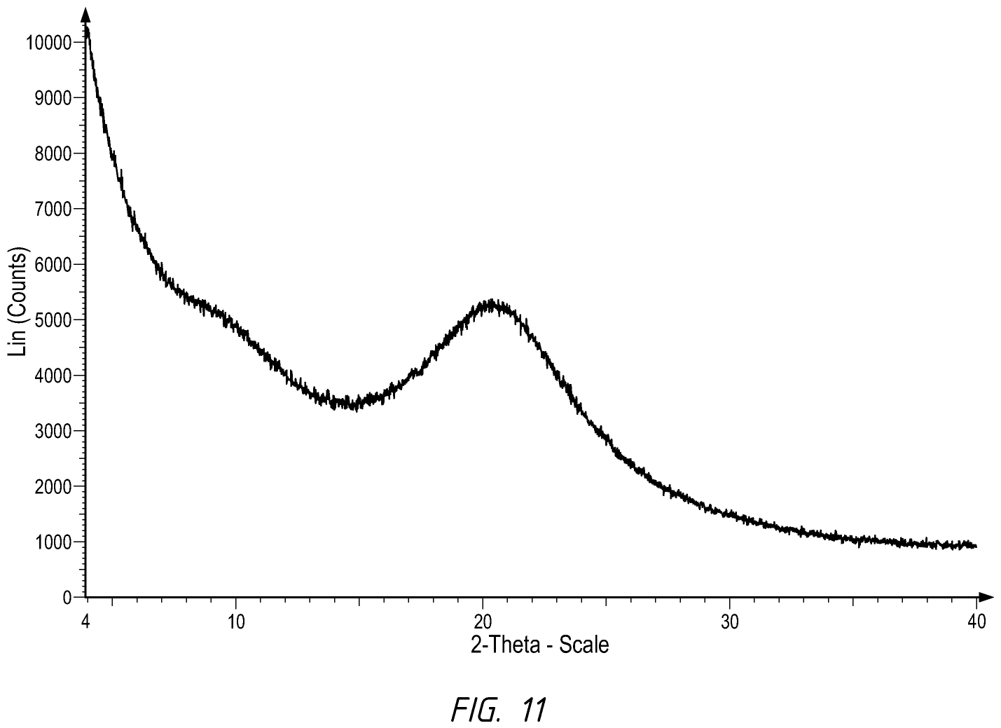

An XRPD pattern of the sample after DVS indicates that the sample is still amorphous, as depicted in FIG. 11.

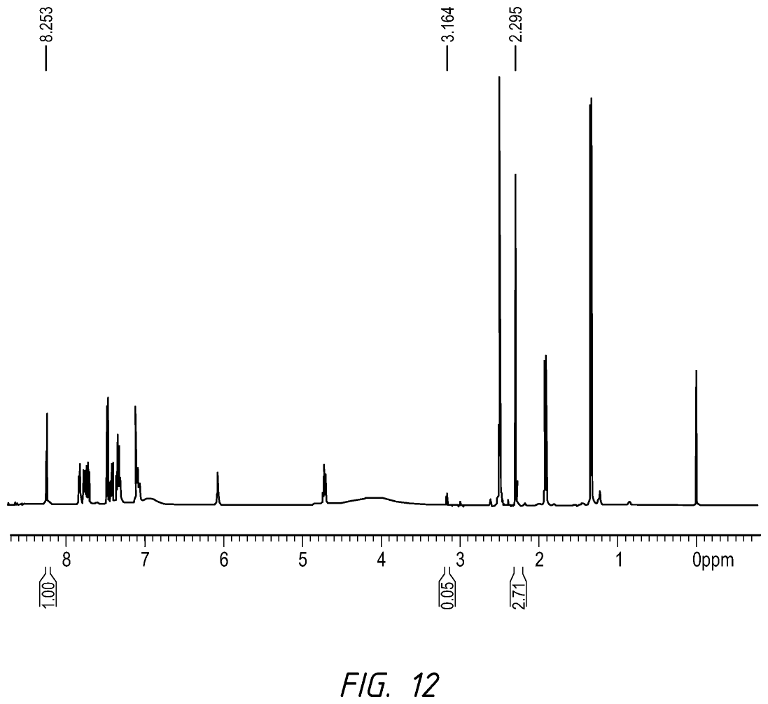

¹H NMR is carried out on a sample of amorphous umbralisib monotosylate prepared according to Example 2 in DMSO-d₆ which indicates an umbralisib tosylate salt with a 1:0.9 ratio of free base to acid, as depicted in FIG. 12. The peak at 8.25 ppm is representative of a single proton in the free base and the peaks at 2.30 ppm are the three protons from p-toluenesulfonic acid. A trace amount (about 0.07%) of methanol is observed at 3.16 ppm.

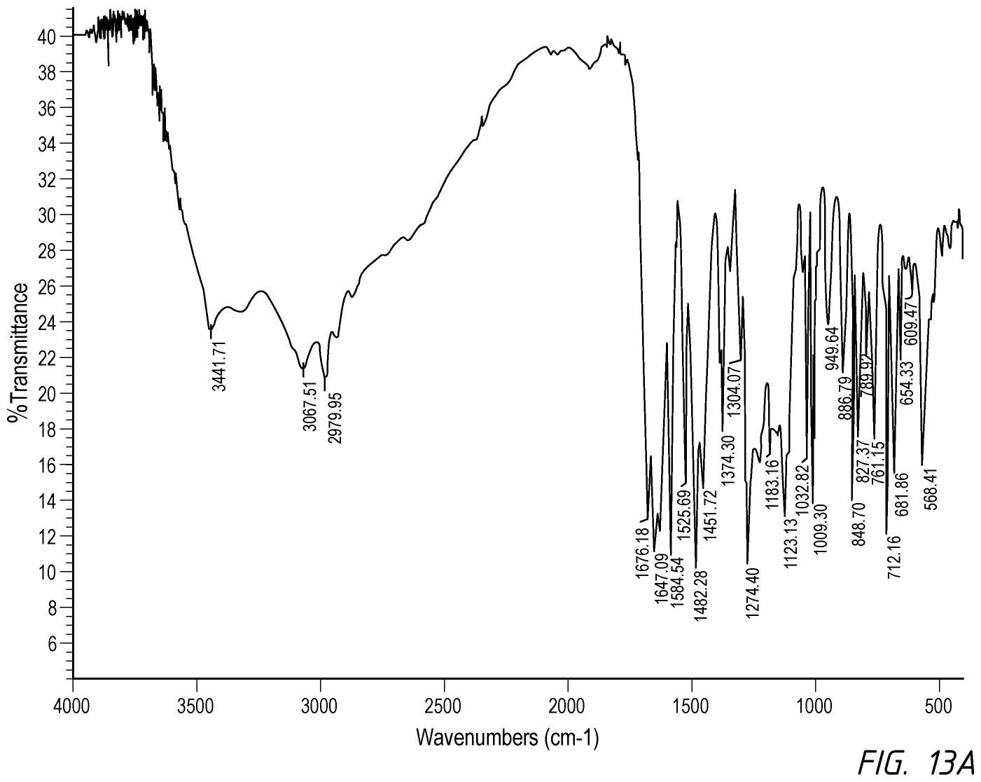

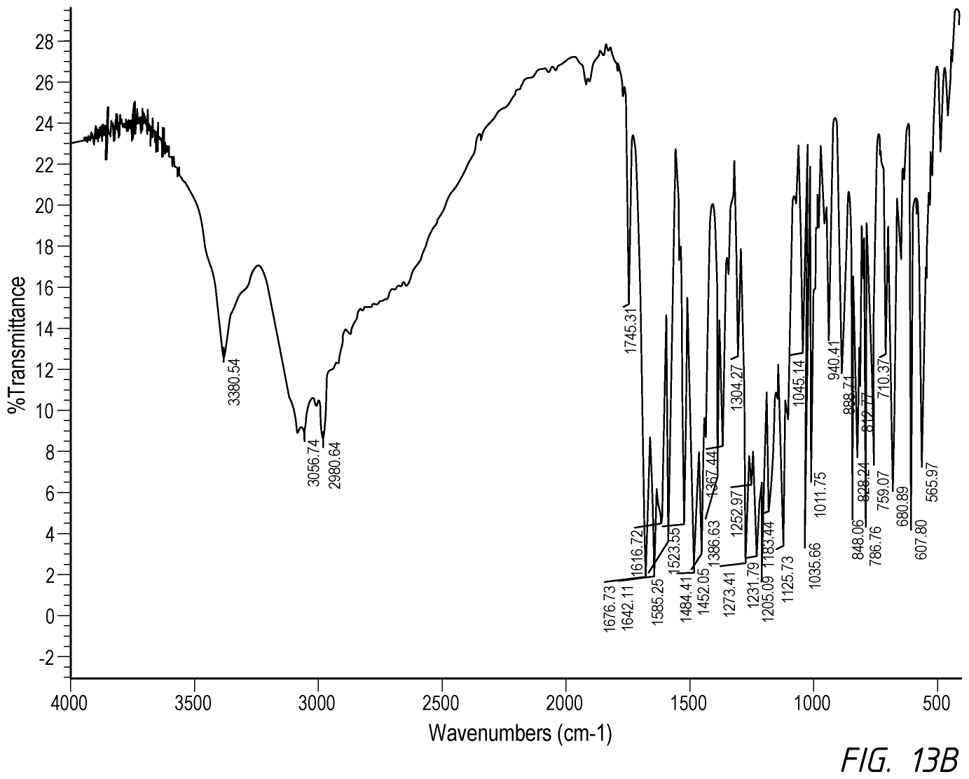

FTIR spectra is collected on amorphous umbralisib monotosylate prepared according to Example 2, as depicted in FIG. 13(a) and on starting crystalline umbralisib tosylate salt, as depicted in FIG. 13(b).

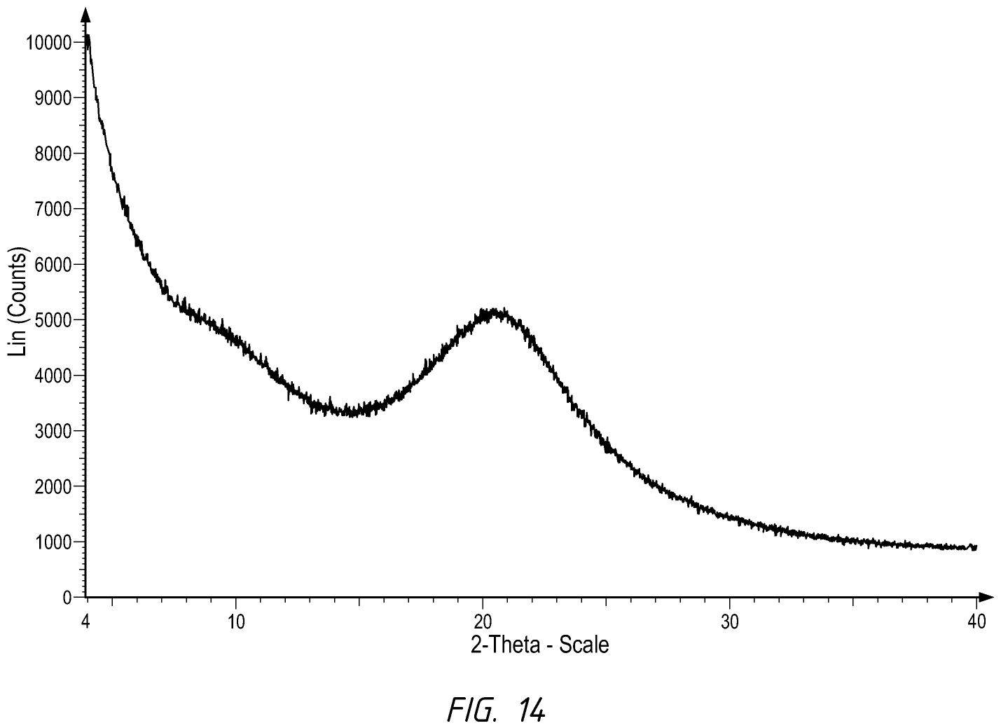

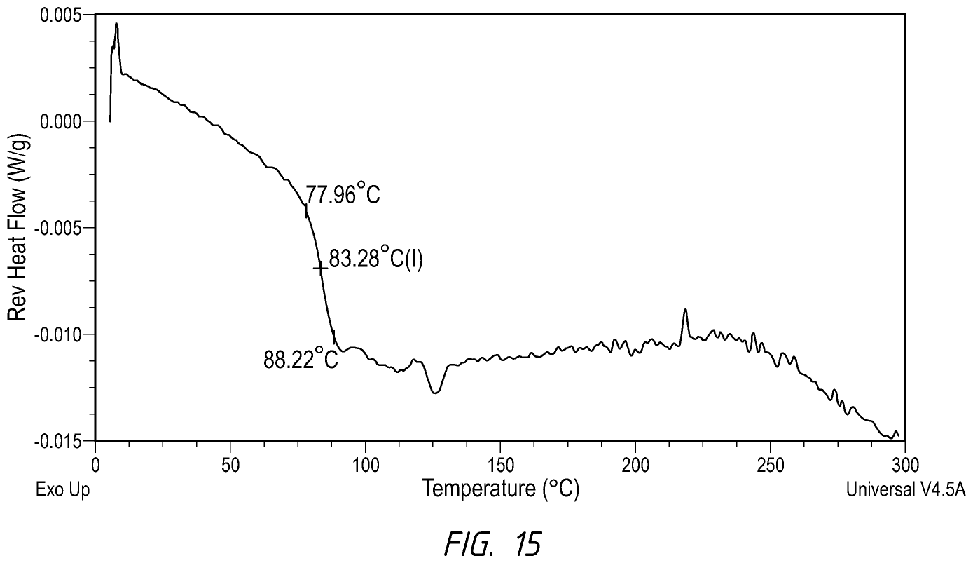

XRPD of amorphous umbralisib monotosylate prepared according to Example 2 after storage at about 40° C. under vacuum conditions for about two weeks indicates that the sample is still amorphous, as depicted in FIG. 14. Further, mDSC of amorphous umbralisib monotosylate after storage at about 40° C. under vacuum conditions for about two weeks indicates that the T_{g} is increased to about 83° C., as depicted in FIG. 15.

### Example 3

### Solution Preparation of Amorphous Umbralisib Monotosylate from Umbralisib Free Base and p-Toluenesulfonic Acid

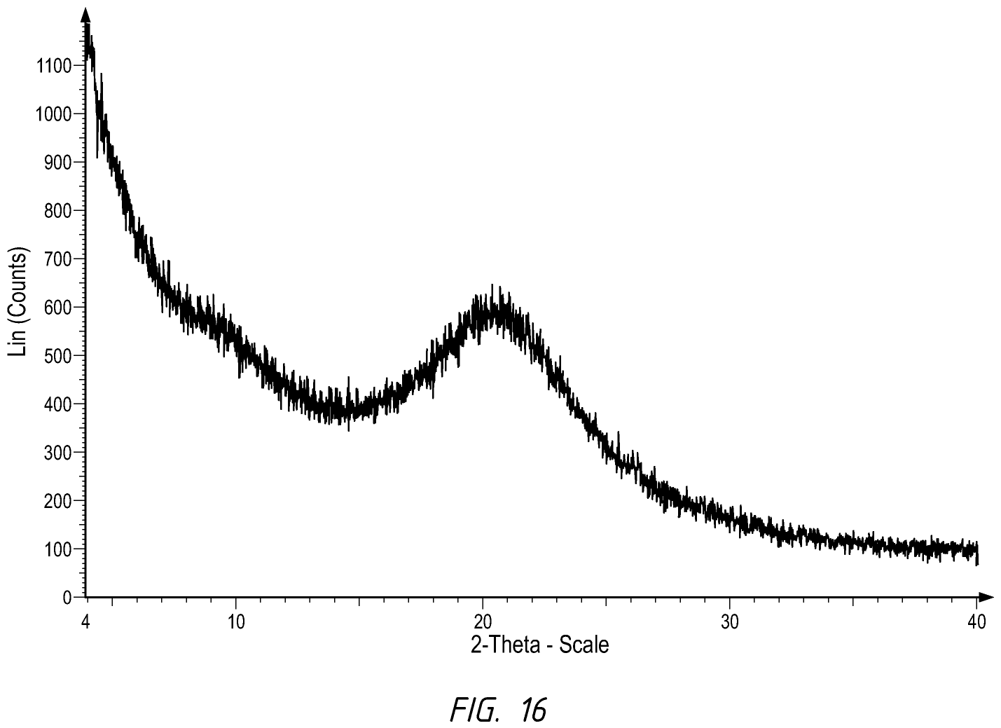

Umbralisib free base and p-toluenesulfonic acid are each separately dissolved in MeOH. Specifically, about 72 mg of umbralisib free base is dissolved in about 3 mL of MeOH at about 50° C. and about 24 mg of p-toluenesulfonic acid is dissolved in about 0.25 mL of MeOH at about 50° C. The two solutions are mixed and stirred at room temperature for about 1 hr and then at about 4° C. overnight. The solution is transferred to a vacuum oven at about 40° C. overnight to evaporate the MeOH. Amorphous umbralisib monotosylate, identified by XRPD, is obtained. FIG. 16 is a representative XPRD pattern for amorphous umbralisib monotosylate prepared according to Example 3.

1. Amorphous umbralisib monotosylate.

2. A process for preparing an amorphous form of umbralisib monotosylate comprising dry grinding a crystalline form of umbralisib tosylate salt to yield amorphous umbralisib monotosylate.

3. The process of claim 2 wherein the dry grinding occurs for about 3 minutes.

4. The process of claim 2 wherein the crystalline form of umbralisib tosylate salt is dried before dry grinding.

5. An amorphous form of umbralisib monotosylate prepared by the process of claim 2.

6. A process for preparing an amorphous form of umbralisib monotosylate comprising:
a. dissolving crystalline umbralisib tosylate salt in a solvent; and
b. evaporating the solvent to yield amorphous umbralisib monotosylate.

7. The process according to claim 6, wherein the solvent is an alcoholic solvent.

8. The process according to claim 7, wherein the alcoholic solvent is methanol.

9. The process according to claim 6, wherein the crystalline umbralisib tosylate salt is dissolved in the solvent at about 50° C.

10. The process according to claim 6, wherein the evaporation occurs under vacuum in an oven at about 40° C.

11. The process according to claim 10, wherein the evaporation occurs overnight.

12. An amorphous form of umbralisib monotosylate prepared by the process of claim 6.

13. A process for preparing an amorphous form of umbralisib monotosylate comprising:
a) forming umbralisib monotosylate in solution; and
b) evaporating the solution to yield amorphous umbralisib monotosylate.

14. The process of claim 13 wherein forming umbralisib monotosylate in solution comprises dissolving umbralisib free base and p-toluenesulfonic acid in a solvent and stirring for a period of time.

15. The process of claim 14 wherein the umbralisib free base and p-toluenesulfonic acid are each dissolved separately in the solvent and then mixed together.

16. The process of claim 14, wherein the solvent is a C1-3 alcohol.

17. The process of claim 16, wherein the C1-3 alcohol is methanol.

18. The process of claim 14, wherein the dissolving occurs at about 50° C.

19. The process of claim 14, wherein the stirring occurs at room temperature for a first period of time and at about 4° C. for a second period of time.

20. The process of claim 13, wherein the evaporating occurs in a vacuum oven at about 40° C. overnight.

21. The process of claim 14, wherein the umbralisib free base and p-toluenesulfonic acid are present in a ratio of about 1:1.

22. An amorphous form of umbralisib monotosylate prepared by the process of claim 13.

23. A pharmaceutical composition comprising a pharmaceutically effective amount of the amorphous umbralisib monotosylate of claim 1 and a pharmaceutically acceptable excipient.

24. A method of treating disease in a patient comprising administering the pharmaceutical composition according to claim 23 to a patient in need thereof.

25. The method of treating disease according to claim 24, wherein the disease is a hematologic malignancy.
